(19) United States
(12) Patent Application Publication
(10) Pub. No.: US 2010/0292509 A1
(43) Pub. Date: Nov. 18, 2010

(54) VEGFC PRODUCTION PROMOTER

(75) Inventors: Kentaro Kajiya, Kanagawa (JP); Eriko Taguchi, Kanagawa (JP)

Correspondence Address: FOLEY AND LARDNER LLP;SUITE 500, 3000 K STREET NW, WASHINGTON, DC, 20007, US

(73) Assignee: Shiseido Company, Ltd

(21) Appl. No.: 12/735,470

(22) PCT Filed: Jan. 16, 2009
(86) PCT No.: PCT/JP2009/050580

§ 371 Date: Jul. 20, 2010

(30) Foreign Application Priority Data
Jan. 21, 2008 (JP) 2008-010675

(51) Int. Cl. C07C 237/28

(52) U.S. Cl. 564/164

(57) ABSTRACT

A bloating ameliorant, a lymphatic vessel activator and a VEGFC production promoter comprising a tranexamic acid amide derivative and/or salt thereof.

### TECHNICAL FIELD

The present invention relates to an ameliorant for bloating, lymphedema, wrinkles or obesity by inducing expression of vascular endothelial growth factor C (VEGFC) and activating lymphatic vessel function.

### BACKGROUND ART

The skin's vascular system is present in the dermis, and is composed of blood vessels and lymphatic vessels. Tissue fluids that have migrated outside blood vessels must again return to veins to maintain homeostasis. Veins of the skin efficiently send blood to the center of the body. However, veins themselves lack the ability to incorporate tissue fluids. Consequently, tissue incorporating tissue fluids, namely lymphatic vessels, have come to be understood to be essential structures in the skin as well.

Lymphatic vessels play an important role in maintaining the microenvironment surrounding cells in a constant state by recovering unnecessary substances present in the skin as well as moisture and proteins that constantly leak from blood vessels. In addition, they are also thought to play a role that resists infectious factors and external factors from the exterior through transport of T lymphocytes.

Known examples of symptoms associated with the pathology of lymphatic vessel dysfunction include bloating and lymphedema (Non-Patent Document 1: Jussila, L. & Alitalo, K. (2002) Physiol. Rev. 82, 673-700). In addition, the functions of lymphatic vessels are also known to play an important role not only with respect to bloating, but also in photoaging (wrinkle formation) of skin accompanying exposure to ultraviolet rays (Non-Patent Document 2: Kajiya, K. & Detmar, M. (2006) J. Invest. Dermatol., 126, 919-21).

On the basis of research conducted thus far, VEGFR-3 has been identified as a transmembrane receptor specifically present in lymphatic vessels, and VEGFC and VEGFD have been found to be ligands thereof. VEGFC activates lymphatic vessel function by promoting growth and migration of lymphatic vascular endothelial cells as well as lumen formation by acting on lymphatic vessels (Non-Patent Document 1: Jussila, L. & Alitalo, K. (2002) Physiol. Rev. 82, 673-700). In addition, studies have been conducted regarding the potential of gene therapy involving introduction of VEGFC for edema as a pathological state of bloating (Non-Patent Document 3: Saaristo, A., Tammela, T., Timonen J., Yla-Herttuala, S., Tukiainen, E., Asko-Seljavaara, S. & Alitalo, K. (2004) Faseb. J., 18, 1707-9).

More recently, genetically mutant mice bred to cause functional abnormalities of lymphatic vessels are known to present with obesity as they mature. Findings have been obtained regarding the mechanism by which abnormalities in lymphatic vessel formation and function cause obesity that indicate that lymph flowing through lymphatic vessels differentiates into fat of preadipocytes (Non-Patent Document 4: Harvey, N. L. et al., Nat. Genet. 2005, 37, 1072-81). In other words, functional abnormalities of lymphatic vessels have been reported to cause leakage of lymph outside lymphatic vessels, which then differentiates into fat and eventually leads to formation of obesity. Thus, a VEGFC promoter is expected to be a therapeutic drug for the prevention of obesity that results in functional lymphangiogenesis.

Members of the VEGF gene family consist of VEGF-A to VEGF-E. Among these, VEGFB and VEGFE have been identified as factors that only act on blood vessels. VEGFA is known to be a factor that is present in skin and but conversely acts to exacerbate the function of lymphatic vessels (Non-Patent Document 5: Nagy, J. A., Vasile, E., Feng, D., Sundberg, C., Brown, L. F., Detmar, M. J., Lawitts, J. A., Benjamin, L., Tan, X., Manseau, E. J., Dvorak, A. M. & Dvorak, H. F. (2002) J. Exp. Med. 196, 1497-506).

In the skin, although VEGFD is reported to only be present in minute amounts in the dermis, since VEGFD knockout mice do not present with any morphological or functional abnormalities in skin lymphatic vessels, they are considered to not be essential factors for the formation of skin lymphatic vessels (Non-Patent Document 6: Baldwin, M. E., Halford, M. M., Roufail, S., Williams, R. A., Hibbs, M. L., Grail, D., Kubo, H., Stacker, S. A. & Achen, M. G. (2005) Mol. Cell. Biol. 25, 2441-9). On the other hand, VEGFC in the skin is strongly expressed in the epidermis. Mice that highly express VEGFC in the epidermis demonstrate increased numbers of lymphatic vessels in the dermis (Non-Patent Document 7: Jeltsch, M., Kaipainen, A., Joukov, V., Meng, X., Lakso, M., Rauvala, H., Swartz, M., Fukumura, D., Jain, R. K. & Alitalo, K. (1997) Science 276, 1423-5), while on the other hand, when the effects of VEGFC in the epidermis were blocked by highly expressing neutralizing antibody of a receptor of VEGFC in the form of VEGFR-3 in the epidermis, the number of lymphatic vessels in the dermis decreased dramatically and findings indicating the formation of edema were obtained (Non-Patent Document 8: Makinen, T., Jussila, L., Veikkola, T., Karpanen, T., Kettunen, M. I., Pulkkanen, K. J., Kauppinen, R., Jackson, D. G., Kubo, H., Nishikawa, S., Yla-Herttula, S. & Alitalo, K. (2001) Nat. Med. 7, 199-205). On the basis of these findings, it has been determined that the function of lymphatic vessels present in the skin dermis is controlled by VEGFC expressed in the skin, and that bloating (edema) occurs when VEGFC no longer functions.

Therefore, compounds that promote expression of VEGFC were screened as a method for eliminating bloating and the like by activating lymphatic vessels.
Non-Patent Document 1: Physiol. Rev. 82, 673-700 (2002)
Non-Patent Document 2: J. Invest. Dermatol. 126, 919-21 (2006)
Non-Patent Document 3: Faseb. J. 18, 1707-9 (2004)
Non-Patent Document 4: Nat. Genet. 37, 1072-81 (2005)
Non-Patent Document 5: J. Exp. Med. 196, 1497-506 (2002)
Non-Patent Document 6: Mol. Cell. Biol. 25, 2441-9 (2005)
Non-Patent Document 7: Science 276, 1423-5 (1997)
Non-Patent Document 8: Nat. Med. 7, 199-205 (2001)

### DISCLOSURE OF THE INVENTION

### Problems to be Solved by the Invention

An object of the present invention is to find a novel drug that is effective for prevention or control of bloating, lymphedema, wrinkle formation or obesity from the viewpoint of activating lymphatic vessel function by inducing expression of VEGFC.

### Means for Solving the Problems

The inventors of the present invention found that a tranexamic acid amide derivative and/or salt thereof induces expression of VEGFC by screening numerous compounds. In other words, a tranexamic acid amide derivative and/or salt thereof that induces expression of VEGFC was found to be extremely effective for preventing or inhibiting bloating, lymphedema, wrinkle formation caused by photoaging of skin accompanying exposure to ultraviolet light or obesity by promoting activation of lymphatic vessel function.

The constitution of the present invention is as described below.

[a] A bloating ameliorant comprising a tranexamic acid amide derivative represented by the following general formula (I):

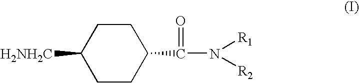

wherein, R₁ and R₂ represent hydrogen atoms, linear or branched alkyl groups having 1 to 18 carbon atoms, cycloalkyl groups having 5 to 8 carbon atoms, benzyl groups or groups represented by the following general formula (II):

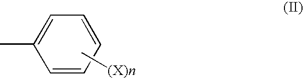

wherein, X represents a lower alkyl group, lower alkoxy group, hydroxy group, amino group or halogen atom and n=0 to 3, and
R₁ and R₂ may be the same or different, and/or a salt thereof.
[b] A lymphedema ameliorant comprising the tranexamic acid amide derivative and/or salt thereof described in [a].
[c] A wrinkle ameliorant comprising the tranexamic acid amide derivative and/or salt thereof described in [a].
[d] An obesity ameliorant comprising the tranexamic acid amide derivative and/or salt thereof described in [a].
[e] A lymphatic vessel neogenesis and/or function promoter comprising the tranexamic acid amide derivative and/or salt thereof described in [a].
[f] A VEGFC production promoter comprising the tranexamic acid amide derivative and/or salt thereof described in [a].
[g] The lymphatic vessel neogenesis and/or function promoter described in [e] that prevents or inhibits bloating.
[h] The VEGFC production promoter described in [f] that prevents or inhibits bloating.
[i] The lymphatic vessel neogenesis and/or function promoter described in [e] that prevents or inhibits lymphedema.
[j] The VEGFC production promoter described in [f] that prevents or inhibits lymphedema.
[k] The lymphatic vessel neogenesis and/or function promoter described in [e] that prevents or inhibits wrinkle formation.
[l] The VEGFC production promoter described in [f] that prevents or inhibits wrinkle formation.
[m] The lymphatic vessel neogenesis and/or function promoter described in [e] that prevents or inhibits obesity.
[n] The VEGFC production promoter described in [f] that prevents or inhibits obesity.
[o] A method for inhibiting bloating comprising applying the lymphatic vessel neogenesis and/or function promoter described in [e] to the skin.
[p] A method for inhibiting bloating comprising applying the VEGFC production promoter described in [f] to the skin.
[q] A method for inhibiting wrinkle formation comprising applying the lymphatic vessel neogenesis and/or function promoter described in [e] to the skin.
[r] A method for inhibiting wrinkle formation comprising applying the VEGFC production promoter described in [f] to the skin.

### BRIEF DESCRIPTION OF THE DRAWINGS

FIG. 1 shows the effects of promoting expression of VEGFC of tranexamic acid methylamide hydrochloride.

### BEST MODE FOR CARRYING OUT THE INVENTION

Definitions: The term “bloating” in the present description is used with the same meaning as “edema”, and refers to a state in which tissue fluid or lymph accumulates in cells, intercellular space or body cavities for some reason, and which is observed in subcutaneous tissue. Moreover, the term “lymphedema” in the present description refers to a state in which tissue fluid accumulates in peripheral portions of the body due to occlusion of lymphatic vessels.

Screening of drugs that induce lymphatic vessel activating factor in the form of VEGFC was carried out as described in detail in the following examples by allowing various candidate drugs to act on cell line HaCat, which consists of cultured cells derived from human epidermis, quantifying VEGFC, and selecting drugs having VEGFC induction activity as effective drugs by using the amount of VEGFC as an indicator of VEGFC induction activity.

As a result, a tranexamic acid derivative and/or salt thereof was found to induce VEGFC.

The tranexamic acid amide derivative as claimed in the present invention is a compound represented by the following general formula (I):

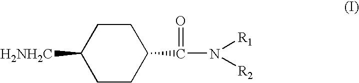

wherein, R₁ and R₂ represent hydrogen atoms, linear or branched alkyl groups having 1 to 18 carbon atoms, cycloalkyl groups having 5 to 8 carbon atoms, benzyl groups or groups represented by the following general formula (II):

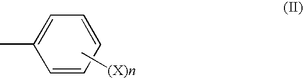

wherein, X represents a lower alkyl group, lower alkoxy group, hydroxy group, amino group or halogen atom and n=0 to 3, and
R₁ and R₂ may be the same or different.

The tranexamic acid amide derivative represented by general formula (I) as claimed in the present invention is a known substance, and can be easily synthesized by a known method such as the method described in ActPharm. Suecica, 7, 441 (1970) or J. Med. Chem., 15, 247 (1972). Namely, after protecting the amino group of tranexamic acid with an appropriate protecting group such as a benzyloxycarbonyl group, an amide form of protected tranexamic acid is produced by allowing an amine component to react with the protected form or reactive derivative of the protected form. An acid halide such as an acid chloride or acid bromide or a mixed acid anhydride and the like are preferable as a reactive derivative of the protected form. Subsequently, the protecting group is eliminated by contact reduction and the like to produce an amide derivative of tranexamic acid.

In addition, the tranexamic acid amide derivative as claimed in the present invention can be converted to an inorganic salt or organic salt by a known method. Although there are no particular limitations on the salt used in the present invention, examples include inorganic salts such as hydrochlorides, sulfates, phosphates, hydrobromides, sodium salts, potassium salts, magnesium salts, calcium salts or ammonium salts. Examples of organic salts include acetates, lactates, maleates, fumarates, tartrates, citrates, methanesulfonates, p-toluenesulfonates, triethanolamine salts, diethanolamine salts and amino acid salts. Hydrochlorides are used preferably.

The tranexamic acid amide derivative and/or salt thereof as claimed in the present invention is extremely effective for promoting the formation and function of lymphatic vessels. Examples of symptoms associated with lymphatic vessel dysfunction include not only bloating and lymphedema, but also photoaging of skin accompanying exposure to ultraviolet light (such as wrinkle formation) and obesity. Thus, the tranexamic acid amide derivative and/or salt thereof as claimed in the present invention is effective for preventing and inhibiting bloating and lymphedema as well as photoaging of skin accompanying exposure to ultraviolet light and obesity. Moreover, the tranexamic acid amide derivative and/or salt thereof is also considered to be effective for treatment of congenital lymphedema.

Photoaging of skin typically refers to changes in skin appearance and function observed as a result of repeated exposure to sunlight. Ultraviolet (UV) light, which is a composite element of sunlight, and particularly intermediate UV light (referred to as UVB having a wavelength of 290 to 320 nm) is the main cause of photoaging. The level of exposure to UVB required for causing photoaging is currently unknown. However, repeated exposure to UVB at a level that causes erythema or sunburn normally leads to photoaging. Photoaging can be identified clinically by rough skin, wrinkle formation, colored spots, soil-colored skin, formation of sagging skin, onset of angiotelectasia, mole formation, onset of purpura, increased susceptibility to skin injury, hypertrophy, formation of areas of fibrotic depigmentation, and the occurrence of premalignant and malignant tumors. Photoaging normally occurs in skin routinely exposed to sunlight, such as skin of the face, ears, head, neck and hands.

The tranexamic acid amide derivative and/or salt thereof in the bloating ameliorant, lymphedema ameliorant, wrinkle ameliorant, obesity ameliorant, lymphatic vessel neogenesis and/or function promoter or VEGFC production promoter of the present invention is incorporated at 20 to 0.001% by weight, and preferably at 7 to 0.01% by weight, based on the total weight of the drug.

In addition, a component other than the aforementioned essential component routinely used in cosmetics, pharmaceuticals and other skin preparations for external use, such as whiteners, moisturizers, antioxidants, oily components, ultraviolet absorbers, surfactants, thickeners, alcohols, powdered components, colorants, aqueous components, water or various types of skin nutrients, can be suitably incorporated in the bloating ameliorant, lymphedema ameliorant, wrinkle ameliorant, obesity ameliorant, lymphatic vessel neogenesis and/or function promoter or VEGFC production promoter of the present invention as necessary.

In addition, metal chelating agents such as disodium edetate, trisodium edetate, sodium citrate, sodium polyphosphate, sodium metaphosphate or gluconic acid, drugs such as pantothenyl ethyl ether, caffeine, tannin, verapamil, glycyrrhiza extract, glabridin, rosewood fruit hot water extract, various types of herbal medicines, yeast extract, tocopherol acetate, glycyrrhizic acid, and derivatives or salts thereof, whiteners such as allantoin, vitamin C, magnesium ascorbyl phosphate, ascorbyl glucoside, albutin, kojic acid or potassium 4-methoxysilicylate, sugars such as Glucose, fructose, mannose, saccharose or trehalose, vitamin A substances such as retinoic acid, retinol, retinol acetate or retinol palmitate, L-serine, hyaluronic acid and derivatives thereof can also be suitably incorporated.

The bloating ameliorant, lymphedema ameliorant, wrinkle ameliorant, obesity ameliorant, lymphatic vessel neogenesis and/or function promoter or VEGFC production promoter of the present invention may be used in any type of conventional skin preparation for external use, examples of which include ointments, creams, milky liquids, lotions, packs or bath oils, may be used for aesthetic purposes, and there are no particular limitations on the form thereof.

### EXAMPLES

HaCaT cells, which are cultured cells derived from human epidermis (German Cancer Research Center, Heidelberg, Germany), were cultured in DMEM containing 10% FBS and seeded into a 24-well plate at 15×10⁴ cells per well. After about 24 hours, the medium was replaced with serum-free DMEM followed by culturing for 24 hours. Subsequently, a solution of a candidate drug dissolved in water to a concentration of 7% by weight was added in an amount equal to 1/100, 1/1000 or 1/10000 the amount of culture liquid followed by additional culturing. 48 hours after adding the drug, the culture supernatant was recovered and the amount of VEGFC in the culture supernatant was quantified by ELISA (R&D Systems) in accordance with the protocol. In addition, Alamar Blue (TREK Diagnostic Systems) was added to the cells following recovery of culture supernatant followed by measurement of the number of cells as fluorescence intensity (Ex: 544 nm, Em: 590 nm). The amount of VEGFC expressed per cell was determined by dividing the amount of VEGFC in the culture supernatant by the fluorescence value of the Alamar Blue. A solvent in the form of 70% EtOH was added to the cells instead of drug for use as a control, and the value thereof was assigned a value of 100. In addition, 5% FBS was used as a positive control.

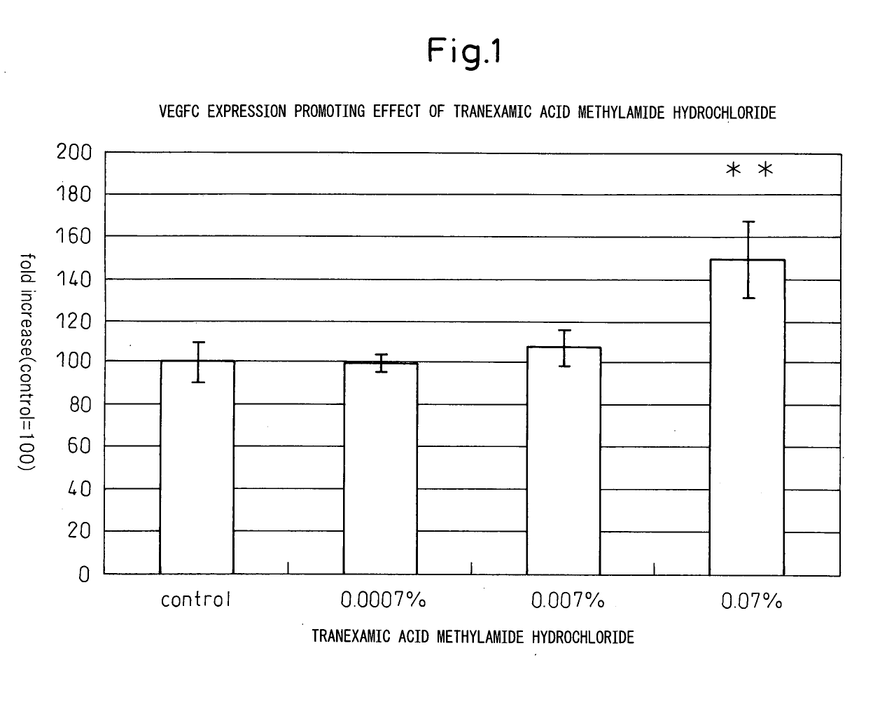

On the basis of the above screening results, tranexamic acid amide derivative and/or salt thereof, and particularly tranexamic acid methylamide hydrochloride, demonstrated dose-dependent VEGFC induction activity that exceeded that of the control (FIG. 1).

### Prescription Example 1

### O/W Cream

| Prescription | (wt %) |
|---|---|
| Liquid paraffin | 3 |
| Vaseline | 1 |
| Dimethylpolysiloxane | 1 |
| Stearyl alcohol | 1.8 |
| Behenyl alcohol | 1.6 |
| Macadamia nut oil | 2 |
| Hydrogenated palm oil | 3 |
| Squalane | 6 |
| Stearic acid | 2 |
| Cholesterol hydroxystearate | 0.5 |
| Cetyl 2-ethylhexanoate | 4 |
| Polyoxyethylene hydrogenated castor oil | 0.5 |
| Self-emulsifying glyceryl monostearate | 3 |
| Tocopherol acetate | 0.1 |
| Retinol | 0.01 |
| (Dimethicone/vinyl dimethicone cross | 3 |
| polymer (trade name: KSG-16, Shin-Etsu | |
| Chemical) | |
| 4-t-butyl-4′-methoxydibenzoylmethane | 0.05 |
| Paraoxybenzoic acid ester | As |
| | suitable |
| Glycerol mono-2-ethylhexanoyl | 0.05 |
| diparamethoxycinnamic acid | |
| Potassium hydroxide | 0.15 |
| Sodium hexametaphosphate | 0.05 |
| Trimethylglycerin | 2 |
| Glycerin | 8 |
| Dipropylene glycol | 5 |
| 2-L-ascorbic acid α-tocopherol phosphoric | 1 |
| acid diester potassium salt | |
| (Ammonium acryloyldimethyl taurate/VP) | 0.1 |
| copolymer (trade name: Aristoflex AVC, | |
| Clariant) | |
| Valerian extract | 0.1 |
| Tranexamic acid methylamide hydrochloride | 1 |
| L-serine | 0.1 |
| Biodyne EMPP | 0.1 |
| Trisodium edetate | 0.05 |
| Carboxyvinyl polymer | 0.05 |
| Flaked inorganic composite powder (talc | 10 |
| coated with titanium oxide, aluminum oxide | |
| and silicic anhydride, trade name: Coverleaf | |
| AR-80, Catalysts and Chemicals Industries) | |
| Titanium-mica | 0.1 |
| Colorant | As |
| | suitable |
| Purified water | Remainder |

### Prescription Example 2

### W/O Cream

| Prescription | (wt %) |
|---|---|
| Dimethylpolysiloxane | 3 |
| Decamethylcyclopentasiloxane | 13 |
| Dodecamethylcyclohexasiloxane | 5 |
| Polyoxyethylene-methylpolysiloxane | 1 |
| copolymer | |
| Pullulan 3-tris(trimethylsiloxy) | 1 |
| silylpropyl carbamate | |
| Dow Corning 9040 Silicone Elastomer Blend | 5 |
| (decamethylcyclopentasiloxane blend of | |
| dimethicone crosspolymer, Dow Corning) | |
| Retinol acetate | 0.1 |
| Paraoxybenzoic acid ester | As |
| | suitable |
| L-menthol | As |
| | suitable |
| Trimethylsiloxysilicic acid | 2 |
| Ethanol | 2 |
| Glycerin | 3 |
| Dipropylene glycol | 5 |
| Polyethylene glycol 6000 | 5 |
| Sodium hexametaphosphate | 0.05 |
| Tocopherol acetate | 0.1 |
| Caffeine | 0.1 |
| Apricot kernel extract | 0.1 |
| Tranexamic acid ethylamide hydrochloride | 0.5 |
| Erythritol | 0.1 |
| Witch hazel extract | 0.1 |
| Carrot extract | 0.1 |
| Biodyne EMPP | 1 |
| Trisodium edetate | 0.05 |
| Dimorpholinopyridazinone | 0.01 |
| Methylbis(trimethylsiloxy)silylisopentyl | 0.1 |
| trimethoxy cinnamate | |
| Flaked inorganic composite powder (talc | 5 |
| coated with titanium oxide, aluminum oxide | |
| and silicic anhydride, trade name: Coverleaf | |
| AR-80, Catalysts and Chemicals Industries) | |
| Yellow iron oxide | As |
| | suitable |
| Cobalt titanate | As |
| | suitable |
| Dimethyl distearyl ammonium hectorite | 1.5 |
| Polyvinyl alcohol | 0.1 |
| Hydroxyethyl cellulose | 0.1 |
| Sodium acrylate/2-acrylamido-2- | 0.1 |
| methylpropanesulfonate copolymer | |
| (trade name: Simulgel EG, Seppic) | |
| (Ammonium acryloyldimethyl taurate/VP) | 0.1 |
| copolymer (trade name: Aristoflex AVC, | |
| Clariant) | |
| Fragrance | As |
| | suitable |
| Purified water | Remainder |

### Prescription Example 3

### O/W Cream for Wrinkles

| Prescription | (wt %) |
|---|---|
| Liquid paraffin | 8 |
| Vaseline | 3 |
| Dimethylpolysiloxane | 2 |
| Dow Corning 9041 Silicone Elastomer Blend | 0.1 |
| (dimethylpolysiloxane blend of dimethicone | |
| crosspolymer, Dow Corning) | |
| Stearyl alcohol | 3 |
| Behenyl alcohol | 2 |
| Glycerin | 5 |
| Dipropylene glycol | 4 |
| Trehalose | 1 |
| Pentaerythritol tetra(2-ethylhexanoate) | 4 |
| Polyoxyethylene glyceryl monoisostearate | 2 |
| Polyoxyethylene glycerin monostearate | 1 |
| Lipophilic glyceryl monostearate | 2 |
| Citric acid | 0.05 |
| Sodium citrate | 0.05 |
| Potassium hydroxide | 0.015 |
| Oil-soluble glycyrrhiza extract | 0.1 |
| Melilotus extract | 0.1 |
| Tranexamic acid propylamide hydrochloride | 2 |
| Allantoin | 0.1 |
| Retinol palmitate (1 million units) | 0.25 |
| Tocopherol acetate | 0.1 |
| Biodyne EMPP | 5 |
| Paraoxybenzoic acid ester | As |
| | suitable |
| Phenoxyethanol | As |
| | suitable |
| Dibutylhydroxytoluene | As |
| | suitable |
| Trisodium edetate | 0.05 |
| 4-t-butyl-4′-methoxybenzoylmethane | 0.01 |
| 2-ethylhexyl-para-methoxycinnamate | 0.1 |
| β-carotene | 0.01 |
| Polyvinyl alcohol | 0.5 |
| Hydroxyethyl cellulose | 0.5 |
| Carboxyvinyl polymer | 0.05 |
| (Ammonium acryloyldimethyl taurate/VP) | 5 |
| copolymer (trade name: Aristoflex AVC, | |
| Clariant) | |
| Purified water | Remainder |
| Fragrance | As |
| | suitable |

### Prescription Example 4

### Moisturizing O/W Cream

| Prescription | (wt %) |
|---|---|
| Liquid paraffin | 10 |
| Dimethylpolysiloxane | 2 |
| Dow Corning 9045 Silicone Elastomer Blend | 3 |
| (decamethylcyclopentasiloxane blend of | |
| dimethicone crosspolymer, Dow Corning) | |
| Glycerin | 10 |
| 1,3-butylene glycol | 2 |
| Erythritol | 1 |
| Polyethylene glycol 1500 | 5 |
| Squalane | 15 |
| Pentaerythritol tetra(2-ethylhexanoate) | 5 |
| Potassium hydroxide | 0.1 |
| Sodium hexametaphosphate | 0.05 |
| Tocopherol acetate | 0.05 |
| Biodyne EMPP | 0.1 |
| Lamium Album extract | 1 |
| Tranexamic acid cyclohexylamide | 0.8 |
| hydrochloride | |
| Isostearic acid | 0.01 |
| Paraoxybenzoic acid ester | As |
| | suitable |
| Hydroxypropyl methylcellulose | 0.3 |
| Polyvinyl alcohol | 0.1 |
| Carboxyvinyl polymer | 0.2 |
| Sodium acrylate/2-acrylamido-2- | 0.5 |
| methylpropanesulfonate copolymer | |
| (trade name: Simulgel EG, Seppic) | |
| Acrylate/alkyl methacrylate copolymer | 0.1 |
| (Pemulen TR-2) | |
| Flaked inorganic composite powder (talc | 5 |
| coated with titanium oxide, aluminum oxide | |
| and silicic anhydride, trade name: Coverleaf | |
| AR-80, Catalysts and Chemicals Industries) | |
| Purified water | Remainder |

### Prescription Example 5

### W/O Sunscreen

| Prescription | (wt %) |
|---|---|
| Dimethylpolysiloxane | 5 |
| Decamethylcyclopentasiloxane | 10 |
| Trimethylsiloxysilicic acid | 3 |
| Pullulan 3-tris(trimethylsiloxy) | 5 |
| silylpropyl carbamate | |
| Polyoxyethylene-methylpolysiloxane | 3 |
| copolymer | |
| (Dimethicone/vinyl dimethicone cross | 30 |
| polymer (trade name: KSG-16, Shin-Etsu | |
| Chemical) | |
| Dipropylene glycol | 3 |
| Cetyl 2-ethylhexanoate | 1 |
| Silicone-coated fine particulate zinc | 10 |
| oxide (60 nm) | |
| Talc | 1 |
| Silicone-coated fine particulate titanium | 7 |
| oxide (40 nm) | |
| Biodyne EMPP | 1 |
| Hypotaurine | 0.1 |
| Sophora Angustifolia extract | 0.01 |
| Adenosine triphosphate disodium | 0.5 |
| Iris Florentina root extract | 0.1 |
| Tranexamic acid benzylamide hydrochloride | 0.5 |
| Retinol acetate (1.5 million units) | 0.01 |
| Paraoxybenzoic acid ester | As |
| | suitable |
| Phenoxyethanol | As |
| | suitable |
| Trisodium edetate | 0.2 |
| Dimethyl distearyl ammonium hectorite | 1 |
| Polymethyl methacrylate copolymer | 3 |
| spherical powder | |
| Flaked inorganic composite powder (talc | 1 |
| coated with titanium oxide, aluminum oxide | |
| and silicic anhydride, trade name: Coverleaf | |
| AR-80, Catalysts and Chemicals Industries) | |
| Titanated mica | 0.1 |
| Purified water | Remainder |
| Fragrance | As |
| | suitable |

### Prescription Example 6

### O/W Cream

| Prescription | (wt %) |
|---|---|
| Glycerin | 3 |
| Decamethylcyclopentasiloxane | 7 |
| Dimethylpolysiloxane | 5 |
| Polyoxyethylene sorbitan monooleate | 1 |
| (20 E.O.) | |
| Polyethylene glycol 20000 | 0.5 |
| Polyvinyl alcohol | 1 |
| Ethanol | 5 |
| Pullulan 3-tris(trimethylsiloxy) | 3 |
| silylpropyl carbamate | |
| Trimethylsiloxysilicic acid | 0.1 |
| Dimorpholinopyridazinone | 0.1 |
| Potassium hydroxide | 0.12 |
| Titanated mica | 0.1 |
| Dow Corning 9045 Silicone Elastomer Blend | 20 |
| (decamethylcyclopentasiloxane blend of | |
| dimethicone crosspolymer, Dow Corning) | |
| (Ammonium acryloyldimethyl taurate/VP) | 2 |
| copolymer (trade name: Aristoflex AVC, | |
| Clariant) | |
| Sodium acrylate/2-acrylamido-2- | 0.1 |
| methylpropanesulfonate copolymer | |
| (trade name: Simulgel EG, Seppic) | |
| Carbomer | 0.01 |
| Acrylate/alkyl methacrylate copolymer | 0.05 |
| (Pemulen TR-2) | |
| Flaked inorganic composite powder (talc | 5 |
| coated with titanium oxide, aluminum oxide | |
| and silicic anhydride, trade name: Coverleaf | |
| AR-80, Catalysts and Chemicals Industries) | |
| Aluminum oxide | 0.01 |
| Xanthan gum | 0.1 |
| Sodium carboxymethyl cellulose | 0.05 |
| Biodyne EMPP | 5 |
| Tranexamic acid isopropylamide | 0.4 |
| hydrochloride | |
| Shosaikoto extract | 0.1 |
| Retinol palmitate (1 million units) | 0.01 |
| Paraoxybenzoic acid ester | As |
| | suitable |
| Phenoxyethanol | As |
| | suitable |
| Fragrance | As |
| | suitable |
| Ion exchange water | Remainder |

### Prescription Example 7

### W/O Cream

| Prescription | (wt %) |
|---|---|
| Dipropylene glycol | 1 |
| Decamethylcyclopentasiloxane | 5 |
| Dimethylpolysiloxane | 1 |
| Polyoxyethylene-methylpolysiloxane | 2 |
| copolymer | |
| Glyceryl tri(2-ethylhexaonate) | 0.1 |
| Polyoxyethylene sorbitan monostearate | 0.05 |
| (20 E.O.) | |
| Polyethylene glycol 9000 | 2 |
| Polyvinyl alcohol | 0.5 |
| Pullulan 3-tris(trimethylsiloxy) | 5 |
| silylpropyl carbamate | |
| Dimorpholinopyridazinone | 0.1 |
| Potassium hydroxide | 0.03 |
| Aluminum oxide | 0.1 |
| Titanium oxide | 1 |
| Dow Corning 9041 Silicone Elastomer Blend | 40 |
| (dimethylpolysiloxane blend of dimethicone | |
| crosspolymer, Dow Corning) | |
| (Ammonium acryloyldimethyl taurate/VP) | 0.1 |
| copolymer (trade name: Aristoflex AVC, | |
| Clariant) | |
| Sodium acrylate/2-acrylamido-2- | 0.1 |
| methylpropanesulfonate copolymer | |
| (trade name: Simulgel EG, Seppic) | |
| Carbomer | 0.01 |
| Acrylate/alkyl methacrylate copolymer | 0.05 |
| (Pemulen TR-2) | |
| Talc | 1 |
| Flaked inorganic composite powder (talc | 1 |
| coated with titanium oxide, aluminum oxide | |
| and silicic anhydride, trade name: Coverleaf | |
| AR-80, Catalysts and Chemicals Industries | |
| Flaked inorganic composite powder | 1 |
| (sericite coated with titanium oxide, | |
| aluminum oxide and silicic anhydride, | |
| trade name: Coverleaf AR-20121M, Catalysts | |
| and Chemicals Industries) | |
| Spherical Nylon powder | 1 |
| Silicic anhydride | 1 |
| Hydroxyethyl cellulose | 0.1 |
| Biodyne EMPP | 1 |
| Crataegus Cuneata fruit extract | 0.1 |
| Tranexamic acid methylamide hydrochloride | 0.1 |
| Potassium 4-methoxysalicylate | 0.1 |
| Paraoxybenzoic acid ester | As |
| | suitable |
| Phenoxyethanol | As |
| | suitable |
| Fragrance | As |
| | suitable |
| Ion exchange water | Remainder |

### Prescription Example 8

### O/W Cream for Wrinkles and Sagging Skin

| Prescription | (wt %) |
|---|---|
| Glycerin | 5 |
| Decamethylcyclopentasiloxane | 1 |
| Dodecamethylcyclohexasiloxane | 3 |
| Dimethylpolysiloxane | 5 |
| Methylphenylpolysiloxane | 1 |
| Octyl methoxycinnamate | 0.1 |
| Pentaerythritol tetra(2-ethylhexanoate) | 1 |
| Polyoxyethylene sorbitan monolaurate | 0.1 |
| (20 E.O.) | |
| Polyoxyethylene-methylpolysiloxane | 1 |
| copolymer (trade name: Polyether-Modified | |
| Silicone Oil KF-6018, Shin-Etsu Chemical) | |
| Soybean fermentation extract | 3 |
| Polyethylene glycol 6000 | 2 |
| Citric acid | 0.1 |
| Sodium citrate | 0.1 |
| Sodium metaphosphate | 0.05 |
| Pullulan 3-tris(trimethylsiloxy) | 0.1 |
| silylpropyl carbamate | |
| (Dimethicone/vinyl dimethicone cross | 10 |
| polymer (trade name: KSG-16, Shin-Etsu | |
| Chemical) | |
| Dow Corning 9041 Silicone Elastomer Blend | 5 |
| (dimethylpolysiloxane blend of dimethicone | |
| crosspolymer, Dow Corning) | |
| (Ammonium acryloyldimethyl taurate/VP) | 0.1 |
| copolymer (trade name: Aristoflex AVC, | |
| Clariant) | |
| Ammonium acryloyldimethyl taurate/ | 0.05 |
| Beheneth-25 methacrylate crosspolymer | |
| (trade name: Aristoflex HMB, Clariant) | |
| Acrylate/alkyl methacrylate copolymer | 0.05 |
| (Pemulen TR-2) | |
| Acrylic acid amide/2-acrylamido-2- | 0.5 |
| methylpropanesulfonate copolymer | |
| (trade name: Simulgel 600, Seppic) | |
| Ammonium polyacrylate (trade name: | 0.5 |
| Simulgel A, Seppic) | |
| Flaked inorganic composite powder (talc | 1 |
| coated with titanium oxide, aluminum oxide | |
| and silicic anhydride, trade name: Coverleaf | |
| AR-80, Catalysts and Chemicals Industries) | |
| Flaked inorganic composite powder | 1 |
| (sericite coated with titanium oxide, | |
| aluminum oxide and silicic anhydride, | |
| trade name: Coverleaf AR-20121M, Catalysts | |
| and Chemicals Industries) | |
| (Dimethicone/vinyl dimethicone cross | 1 |
| polymer (trade name: BY29-119, Toray | |
| Dow Corning Silicone) | |
| Biodyne EMPP | 1 |
| Apricot kernel extract | 0.1 |
| Tranexamic acid methylamide hydrochloride | 1.2 |
| Adenosine triphosphate | 0.01 |
| Paraoxybenzoic acid ester | As |
| | suitable |
| Phenoxyethanol | As |
| | suitable |
| Fragrance | As |
| | suitable |
| Ion exchange water | Remainder |

### Prescription Example 9

### O/W Milky Liquid

| Prescription | (wt %) |
|---|---|
| Dimethylpolysiloxane | 3 |
| Decamethylcyclopentasiloxane | 4 |
| Pullulan 3-tris(trimethylsiloxy) | 0.5 |
| silylpropyl carbamate | |
| Dow Corning 9040 Silicone Elastomer Blend | 0.5 |
| (decamethylcyclopentasiloxane blend of | |
| dimethicone crosspolymer, Dow Corning) | |
| Ethanol | 5 |
| Glycerin | 6 |
| 1,3-butylene glycol | 5 |
| Polyoxyethylene methyl glucoside | 3 |
| Sunflower oil | 1 |
| Squalane | 2 |
| Potassium hydroxide | 0.1 |
| Sodium hexametaphosphate | 0.05 |
| Hydroxypropyl-β-cyclodextrin | 0.1 |
| Dipotassium glycyrrhizinate | 0.05 |
| Loquat leaf extract | 0.1 |
| Sodium L-glutamate | 0.05 |
| Tranexamic acid methylamide hydrochloride | 2.5 |
| Hyaluronic acid | 0.001 |
| Lavender oil | 0.1 |
| Vitamin E | 0.1 |
| Rehmannia root extract | 0.1 |
| Hypotaurine | 0.01 |
| Thiotaurine | 0.1 |
| Mortierella oil | 0.5 |
| Albutin | 3 |
| Sodium acetylhyaluronate | 0.1 |
| Marjoram extract | 0.1 |
| Biodyne EMPP | 0.1 |
| Dimorpholinopyridazinone | 0.1 |
| Xanthan gum | 0.1 |
| Carboxyvinyl polymer | 0.1 |
| Acrylate/alkyl methacrylate copolymer | 0.1 |
| (Pemulen TR-1) | |
| (Ammonium acryloyldimethyl taurate/VP) | 1 |
| copolymer (trade name: Aristoflex AVC, | |
| Clariant) | |
| Red iron oxide | As |
| | suitable |
| Yellow iron oxide | As |
| | suitable |
| Flaked inorganic composite powder (talc | 1 |
| coated with titanium oxide, aluminum oxide | |
| and silicic anhydride, trade name: Coverleaf | |
| AR-80, Catalysts and Chemicals Industries) | |
| Titanated mica | 0.1 |
| Paraoxybenzoic acid ester | As |
| | suitable |
| Purified water | Remainder |

### Prescription Example 10

### O/W Milky Liquid

| Prescription | (wt %) |
|---|---|
| Dimethylpolysiloxane | 2 |
| (Dimethicone/vinyl dimethicone cross | 15 |
| polymer (trade name: KSG-16, Shin-Etsu | |
| Chemical) | |
| Behenyl alcohol | 1 |
| Batyl alcohol | 0.5 |
| Glycerin | 5 |
| 1,3-butylene glycol | 7 |
| Erythritol | 2 |
| Biodyne EMPP | 5 |
| Silica-coated zinc oxide | 0.1 |
| Valerian extract | 0.1 |
| Tranexamic acid methylamide hydrochloride | 0.1 |
| Green tea extract | 0.1 |
| Hydrogenated palm oil | 3 |
| Squalane | 6 |
| Pentaerythritol tetra(2-ethylhexanoate) | 2 |
| Polyoxyethylene glyceryl isostearate | 1 |
| Polyoxyethylene glyceryl monostearate | 1 |
| Potassium hydroxide | As |
| | suitable |
| Sodium hexametaphosphate | 0.05 |
| Phenoxyethanol | As |
| | suitable |
| Carboxyvinyl polymer | 0.1 |
| Sodium acrylate/2-acrylamido-2- | 0.1 |
| methylpropanesulfonate copolymer | |
| (trade name: Simulgel EG, Seppic) | |
| Purified water | Remainder |

### Prescription Example 11

### O/W Milky Liquid

| Prescription | (wt %) |
|---|---|
| Liquid paraffin | 7 |
| Vaseline | 3 |
| Decamethylcyclopentasiloxane | 2 |
| Pullulan 3-tris(trimethylsiloxy) | 0.1 |
| silylpropyl carbamate | |
| Dow Corning 9041 Silicone Elastomer Blend | 3 |
| (dimethylpolysiloxane blend of dimethicone | |
| crosspolymer, Dow Corning) | |
| Behenyl alcohol | 1 |
| Glycerin | 5 |
| Dipropylene glycol | 7 |
| Polyethylene glycol 1500 | 2 |
| Jojova oil | 1 |
| Isostearic acid | 0.5 |
| Stearic acid | 0.5 |
| Behenic acid | 0.5 |
| Pentaerythritol tetra(2-ethylhexanoate) | 3 |
| Cetyl 2-ethylhexanoate | 3 |
| Glyceryl monostearate | 1 |
| Polyoxyethylene glyceryl monostearate | 1 |
| Potassium hydroxide | 0.1 |
| Sodium hexametaphosphate | 0.05 |
| Stearyl glycyrrhetinate | 0.05 |
| L-arginine | 0.1 |
| Trimethylglycine | 0.1 |
| Ascorbyl-2-glucoside | 0.1 |
| Xylitol | 1 |
| Iris Florentina root extract | 0.1 |
| Tranexamic acid methylamide hydrochloride | 1 |
| Rose extract | 0.1 |
| Tocopherol acetate | 0.1 |
| Sodium acetylated hyaluronate | 0.1 |
| Biodyne EMPP | 1 |
| Trisodium edetate | 0.05 |
| 4-t-butyl-4′-methoxydibenzoylmethane | 0.1 |
| 2-ethylhexyl-para-methoxycinnamate | 0.1 |
| Carboxyvinyl polymer | 0.15 |
| (Ammonium acryloyldimethyl taurate/VP) | 0.5 |
| copolymer (trade name: Aristoflex AVC, | |
| Clariant) | |
| Flaked inorganic composite powder (talc | 0.5 |
| coated with titanium oxide, aluminum oxide | |
| and silicic anhydride, trade name: Coverleaf | |
| AR-80, Catalysts and Chemicals Industries) | |
| Titanated mica | 0.1 |
| Paraoxybenzoic acid ester | As |
| | suitable |
| Purified water | Remainder |
| Fragrance | As |
| | suitable |

### Prescription Example 12

### W/O Daytime Milky Liquid

| Prescription | (wt %) |
|---|---|
| Dimethylpolysiloxane | 2 |
| Decamethylcyclopentasiloxane | 25 |
| Dodecamethylcyclohexasiloxane | 10 |
| Polyoxyethylene-methylpolysiloxane | 1.5 |
| copolymer | |
| Pullulan 3-tris(trimethylsiloxy) | 10 |
| silylpropyl carbamate | |
| Dow Corning 9045 Silicone Elastomer Blend | 5 |
| (decamethylcyclopentasiloxane blend of | |
| dimethicone crosspolymer, Dow Corning) | |
| Trimethylsiloxysilicic acid | 1 |
| 1,3-butylene glycol | 5 |
| Squalane | 0.5 |
| Talc | 5 |
| Dipotassium glycyrrhizinate | 0.1 |
| Tocopherol acetate | 0.1 |
| Biodyne EMPP | 0.1 |
| L-serine | 1 |
| Carrot extract | 0.1 |
| Tranexamic acid methylamide hydrochloride | 0.7 |
| Carrot extract | 0.1 |
| Trisodium edetate | 0.05 |
| 4-t-butyl-4′-methoxydibenzoylmethane | 1 |
| 2-ethylhexyl-para-methoxycinnamate | 5 |
| Glycerol mono-2-ethylhexanoyl | 1 |
| diparamethoxycinnamic acid | |
| Silicone-coated fine particulate titanium | 4 |
| oxide (40 nm) | |
| Dimethyl distearyl ammonium hectorite | 0.5 |
| Spherical polyethylene powder | 3 |
| Sodium acrylate/2-acrylamido-2- | 0.1 |
| methylpropanesulfonate copolymer | |
| (trade name: Simulgel EG, Seppic) | |
| Flaked inorganic composite powder (talc | 1 |
| coated with titanium oxide, aluminum oxide | |
| and silicic anhydride, trade name: Coverleaf | |
| AR-80, Catalysts and Chemicals Industries) | |
| Titanated mica | 0.1 |
| Phenoxyethanol | As |
| | suitable |
| Purified water | Remainder |
| Fragrance | As |
| | suitable |

### Prescription Example 13

### Beauty Wash

| Prescription | (wt %) |
|---|---|
| Ethyl alcohol | 5 |
| Glycerin | 1 |
| 1,3-butylene glycol | 5 |
| Polyoxyethylene polyoxypropylene decyl | 0.2 |
| tetradecyl ether | |
| Sodium hexametaphosphate | 0.03 |
| Trimethylglycine | 1 |
| Sodium polyaspartate | 0.1 |
| α-Tocopherol 2-L-Ascorbic acid phosphoric | 0.1 |
| acid diester potassium salt | |
| Thiotaurine | 0.1 |
| Crataegus Cuneata fruit extract | 0.1 |
| Tranexamic acid methylamide hydrochloride | 0.5 |
| Uncaria gambir extract | 0.1 |
| Peppermint extract | 0.1 |
| α-Glucosyl hesperidin | 0.01 |
| Ascorbic acid phosphate magnesium ester | 0.01 |
| Potassium 4-methoxysalicylate | 1 |
| Melilotus extract | 0.1 |
| Biodyne EMPP | 0.01 |
| Trisodium HEDTA | 0.1 |
| Carboxyvinyl polymer | 0.05 |
| Sodium acrylate/2-acrylamido-2- | 0.05 |
| methylpropanesulfonate copolymer | |
| (trade name: Simulgel EG, Seppic) | |
| Potassium hydroxide | 0.02 |
| Phenoxyethanol | As |
| | suitable |
| Purified water | Remainder |
| Fragrance | As |
| | suitable |

### Prescription Example 14

### Beauty Wash

| Prescription | (wt %) |
|---|---|
| Ethanol | 10 |
| Glycerin | 2 |
| Dipropylene glycol | 1 |
| Isostearic acid | 0.1 |
| Poly(oxyethylene-oxypropylene)- | 1 |
| methylpolysiloxane copolymer | |
| Betaine lauryldimethylaminoacetate | 0.1 |
| Citric acid | 0.02 |
| Sodium citrate | 0.08 |
| Sodium hexametaphosphate | 0.01 |
| Hypotaurine | 0.1 |
| Lamium Album extract | 0.1 |
| Tranexamic acid methylamide hydrochloride | 1 |
| Scutellaria root extract | 0.1 |
| Biodyne EMPP | 1 |
| Lavender oil | 0.001 |
| (Ammonium acryloyldimethyl taurate/VP) | 0.01 |
| copolymer (trade name: Aristoflex AVC, | |
| Clariant) | |
| Phenoxyethanol | As |
| | suitable |
| Active hydrogen water | 1 |
| Purified water | Remainder |

### Prescription Example 15

### Pack

| Prescription | (wt %) |
|---|---|
| Ethanol | 10 |
| 1,3-butylene glycol | 6 |
| Polyethylene glycol 4000 | 2 |
| Olive oil | 1 |
| Macadamia nut oil | 1 |
| Phytosteryl hydroxystearate | 0.05 |
| Lactic acid | 0.05 |
| Sodium lactate | 0.1 |
| Disodium L-ascorbate sulfuric acid ester | 0.1 |
| α-Tocopherol 2-L-Ascorbic acid phosphoric | 0.1 |
| acid diester potassium salt | |
| Tocopherol acetate | 0.1 |
| Melilotus extract | 0.1 |
| Tranexamic acid ethylamide hydrochloride | 0.1 |
| Molasses extract | 1 |
| Sodium chondroitin sulfate | 0.1 |
| Biodyne EMPP | 3 |
| Sodium carboxymethyl cellulose | 0.2 |
| Polyvinyl alcohol | 12 |
| (Ammonium acryloyldimethyl taurate/VP) | 2 |
| copolymer (trade name: Aristoflex AVC, | |
| Clariant) | |
| Sodium acrylate/2-acrylamido-2- | 0.1 |
| methylpropanesulfonate copolymer | |
| (trade name: Simulgel EG, Seppic) | |
| Dow Corning 9041 Silicone Elastomer Blend | 3 |
| (dimethylpolysiloxane blend of dimethicone | |
| crosspolymer, Dow Corning) | |
| Paraoxybenzoic acid ester | As |
| | suitable |
| Purified water | Remainder |
| Fragrance | As |
| | suitable |

### Prescription Example 16

### Powdered Solid Foundation

| Prescription | (wt %) |
|---|---|
| Dimethylpolysiloxane | 5 |
| Dow Corning 9045 Silicone Elastomer Blend | 5 |
| (decamethylcyclopentasiloxane blend of | |
| dimethicone crosspolymer, Dow Corning) | |
| Isostearic acid | 0.5 |
| Diisostearyl malate | 3 |
| Glyceryl tri(2-ethylhexanoate) | 1 |
| Sorbitan sesquiisostearate | 1 |
| Spherical PMMA-coated mica | 6 |
| Prism Tone Powder YR | 1 |
| Fine particulate zinc oxide | 0.5 |
| Fine particulate titanium oxide | 2 |
| Synthetic phlogopite | 2 |
| Metal soap-treated talc | 8 |
| Spherical silicic anhydride | 5 |
| Tocopherol acetate | 0.1 |
| δ-tocopherol | 0.1 |
| Apricot kernel extract | 0.1 |
| Tranexamic acid methylamide hydrochloride | 2 |
| Biodyne EMPP | 0.1 |
| Paraoxybenzoic acid ester | As |
| | suitable |
| Methylbis(trimethylsiloxy)silylisopentyl | 1 |
| trimethoxy cinnamate | |
| 2-ethylhexyl-para-methoxycinnamate | 3 |
| Spherical alkyl polyacrylate powder | 6 |
| Methyl hydrogen polysiloxane-coated talc | Remainder |
| Methyl hydrogen polysiloxane-coated | 5 |
| sericite | |
| Methyl hydrogen polysiloxane-coated | 15 |
| titanium oxide | |
| Flaked inorganic composite powder (talc | 10 |
| coated with titanium oxide, aluminum oxide | |
| and silicic anhydride, trade name: Coverleaf | |
| AR-80, Catalysts and Chemicals Industries) | |
| Titanated mica | 1 |
| Methyl hydrogen polysiloxane-coated | 5 |
| pigment (colorant) | |

### Prescription Example 17

### Powdered Solid Foundation

| Prescription | (wt %) |
|---|---|
| Sericin | 0.5 |
| Dimethylpolysiloxane | 2 |
| Methylphenylpolysiloxane | 1 |
| (Dimethicone/vinyl dimethicone cross | 10 |
| polymer (trade name: KSG-16, Shin-Etsu | |
| Chemical) | |
| Squalane | 7 |
| Squalane (plant-based) | 1 |
| Sorbitan sesquiisostearate | 1 |
| Glycerol-modified silicon resin-coated | 16 |
| baked sericite | |
| Glycerol-modified silicon resin-coated | 7 |
| sericite | |
| Yellow iron oxide-coated titanated mica | 0.1 |
| Fine particulate titanium oxide | 5 |
| Talc | 10 |
| Titanium oxide-coated sericite | 0.1 |
| Boron nitride | 2.5 |
| Red iron oxide-coated titanated mica | 0.1 |
| Phytosterol | 0.1 |
| Ascorbyl dipalmitate | 0.1 |
| DL-α-tocopherol acetat | 0.1 |
| D-δ-tocopherol | 0.1 |
| Biodyne EMPP | 0.1 |
| Crataegus Cuneata fruit extract | 0.1 |
| Tranexamic acid methylamide hydrochloride | 0.5 |
| Paraoxybenzoic acid ester | As suitable |
| 2-ethylhexyl-para-methoxycinnamate | 1 |
| Spherical alkyl polyacrylate powder | 8 |
| Methyl hydrogen polysiloxane-coated mica | Remainder |
| Baked methyl hydrogen polysiloxane-coated | 5 |
| iron oxide titanium oxide | |
| Methyl hydrogen polysiloxane-coated | 5 |
| sericite | |
| Methyl hydrogen polysiloxane-coated | 4 |
| titanium oxide | |
| Methyl hydrogen polysiloxane-coated | 5 |
| flaked titanium oxide | |
| Methyl hydrogen polysiloxane-coated | 5 |
| pigment (colorant) | |
| Fragrance | As suitable |

### Prescription Example 18

### Powdered Solid Foundation

| Prescription | (wt %) |
|---|---|
| α-olefin oligomer | 3 |
| Vaseline | 3 |
| Dow Corning 9040 Silicone Elastomer Blend | 3 |
| (decamethylcyclopentasiloxane blend of | |
| dimethicone crosspolymer, Dow Corning) | |
| Macadamia nut extract | 0.1 |
| Sorbitan sesquiisostearate | 1 |
| Alkyl-modified silicon-coated yellow | 2 |
| iron oxide | |
| Alkyl-modified silicon resin-coated red | 1 |
| iron oxide | |
| Alkyl-modified silicon resin-coated black | 0.5 |
| iron oxide | |
| Yellow iron oxide-coated titanated mica | 5 |
| Synthetic phlogopite | 5 |
| Titanium oxide | 1 |
| Zinc oxide | 1 |
| Low-temperature-baked zinc oxide | 4 |
| Baked sericite | 10 |
| Phlogopite | 1 |
| Aluminum oxide | 1 |
| Flaked inorganic composite powder (talc | 15 |
| coated with titanium oxide, aluminum oxide | |
| and silicic anhydride, trade name: Coverleaf | |
| AR-80, Catalysts and Chemicals Industries) | |
| Talc | Remainder |
| Synthetic phlogopite | 5 |
| Crosslinked silicone powder (Torayfil | 10 |
| E-506) | |
| DL-α-tocopherol acetate | 0.1 |
| D-δ-tocopherol | 0.1 |
| Biodyne EMPP | 5 |
| Paraoxybenzoic acid ester | As suitable |
| Apricot kernel extract | 0.1 |
| Tranexamic acid hexylamide hydrochloride | 0.5 |
| 2-ethylhexyl-para-methoxycinnamate | 1 |
| Calcium alginate powder | 1 |
| Fragrance | As suitable |

### Prescription Example 19

### Solid Foundation

| Prescription | (wt %) |
|---|---|
| α-olefin oligomer | 10 |
| Microcrystalline wax | 0.5 |
| Dow Corning 9041 Silicone Elastomer Blend | 3 |
| (dimethylpolysiloxane blend of dimethicone | |
| crosspolymer, Dow Corning) | |
| Sericin | 5 |
| Dimethylpolysiloxane | 15 |
| Methylphenylpolysiloxane | 10 |
| Macadamia nut oil | 0.1 |
| Carnauba wax | 0.1 |
| Glyceryl tri(2-ethylhexanoate) | 7 |
| Cetyl 2-ethylhexanoate | 10 |
| Sorbitan sesquiisostearate | 1.5 |
| Mica | 0.5 |
| Aluminum stearate | 1 |
| Crosslinked silicone powder (Torayfil | 8 |
| E-506) | |
| N-lauroyl-L-lysine | 0.1 |
| D-δ-tocopherol | As |
| | suitable |
| Biodyne EMPP | 0.1 |
| Melilotus extract | 0.1 |
| Tranexamic acid methylamide hydrochloride | 0.3 |
| Red iron oxide | As |
| | suitable |
| Yellow iron oxide | As |
| | suitable |
| Calcium alginate powder | 1 |
| Nylon powder | Remainder |
| Spherical silicic anhydride | 1 |
| Titanium oxide | 1 |

### Prescription Example 20

### Emulsified Foundation

| Prescription | (wt %) |
|---|---|
| Microcrystalline wax | 1 |
| Dimethylpolysiloxane | 15 |
| Decamethylcyclopentasiloxane | 2 |
| Dow Corning 9045 Silicone Elastomer Blend | 3 |
| (decamethylcyclopentasiloxane blend of | |
| dimethicone crosspolymer, Dow Corning) | |
| Pullulan 3-tris(trimethylsiloxy) | 0.5 |
| silylpropyl carbamate | |
| 1,3-butylene glycol | 6 |
| Candelilla wax | 3 |
| Isostearic acid | 1 |
| Ethylene glycol fatty acid ester | 0.1 |
| Lanolin fatty acid octododecyl | 0.5 |
| 2-alkyl-N-carboxymethyl-N-hydroxyethyl | 4 |
| imidazolinium betaine | |
| Titanium oxide | 7.5 |
| Barium sulfate | 5 |
| Titanium oxide | 7 |
| Talc | 3 |
| Silicic anhydride | 4 |
| Crosslinked silicone powder (Torayfil | 0.1 |
| E-506) | |
| Sodium metaphosphate | 0.1 |
| Hydroxypropyl-β-cyclodextrin | 0.1 |
| DL-α-tocopherol acetate | 0.1 |
| Witch hazel extract | 0.1 |
| Peony extract | 0.1 |
| Sodium chondroitin sulfate | 0.1 |
| Sodium hyaluronate | 0.1 |
| Biodyne EMPP | 0.1 |
| Lamium Album extract | 0.1 |
| Tranexamic acid methylamide hydrochloride | 0.1 |
| Paraoxybenzoic acid ester | As |
| | suitable |
| Red iron oxide | As |
| | suitable |
| Yellow iron oxide | As |
| | suitable |
| Black iron oxide | As |
| | suitable |
| Flaked inorganic composite powder (talc | 1 |
| coated with titanium oxide, aluminum oxide | |
| and silicic anhydride, trade name: Coverleaf | |
| AR-80, Catalysts and Chemicals Industries) | |
| Xanthan gum | 0.1 |
| Sodium carboxymethyl cellulose | 0.1 |
| Sodium acrylate/2-acrylamido-2- | 0.5 |
| methylpropanesulfonate copolymer | |
| (trade name: Simulgel EG, Seppic) | |
| Christmas begonia extract | 2 |
| Purified water | Remainder |

### Prescription Example 21

### W/O Foundation

| Prescription | (wt %) |
|---|---|
| Dimethylpolysiloxane | 3 |
| Decamethylcyclopentasiloxane | 10 |
| Polyoxyethylene-methylpolysiloxane | 3 |
| copolymer | |
| Dodecamethylcyclohexasiloxane | 5 |
| Pullulan 3-tris(trimethylsiloxy) | 0.1 |
| silylpropyl carbamate | |
| (Dimethicone/vinyl dimethicone cross | 5 |
| polymer (trade name: KSG-16, Shin-Etsu | |
| Chemical) | |
| Glycerin | 4 |
| 1,3-butylene glycol | 5 |
| Palmitic acid | 0.5 |
| Distearyl dimethyl ammonium chloride | 0.2 |
| Metal soap-treated talc | 2 |
| Crosslinked silicone powder (Torayfil | 0.1 |
| E-506) | |
| Red iron oxide-coated titanated mica | 0.5 |
| N-lauroyl-L-lysine | 2 |
| Sodium L-glutamate | 2 |
| Tocopherol acetate | 0.1 |
| δ-tocopherol | 0.1 |
| Paraoxybenzoic acid ester | As |
| | suitable |
| Phenoxyethanol | 0.2 |
| Spherical Nylon powder | 1 |
| Spherical alkyl polyacrylate powder | 3 |
| Melilotus extract | 0.1 |
| Tranexamic acid methylamide hydrochloride | 2 |
| Biodyne EMPP | 5 |
| Purified water | Remainder |
| Dextrin fatty acid-treated talc | 3 |
| Dextrin fatty acid-treated titanium | 15 |
| dioxide | |
| Dextrin fatty acid-treated yellow iron | 3 |
| oxide | |
| Dextrin fatty acid-treated black iron | 0.5 |
| oxide | |

### Prescription Example 22

### O/W Foundation

| Prescription | (wt %) |
|---|---|
| Dimethylpolysiloxane | 8 |
| Dow Corning 9040 Silicone Elastomer Blend | 20 |
| (decamethylcyclopentasiloxane blend of | |
| dimethicone crosspolymer, Dow Corning) | |
| Behenyl alcohol | 0.5 |
| Batyl alcohol | 0.5 |
| 1,3-butylene glycol | 5 |
| Macadamia nut extract | 0.1 |
| Isostearic acid | 1.5 |
| Stearic acid | 1 |
| Behenic acid | 0.5 |
| Cetyl 2-ethylhexanoate | 5 |
| Polyoxyethylene glycerin monostearate | 1 |
| Self-emulsifying glyceryl monostearate | 1 |
| Yellow iron oxide-coated titanated mica | 2 |
| Titanium oxide | 4 |
| Talc | 0.5 |
| Kaolin | 3 |
| Synthetic phlogopite | 0.1 |
| Crosslinked silicone powder | 0.1 |
| Silicic anhydride | 5 |
| Potassium hydroxide | 0.2 |
| Triethanolamine | 0.8 |
| DL-α-tocopherol acetate | 0.1 |
| Sodium hyaluronate | 0.1 |
| Biodyne EMPP | 0.1 |
| Iris Florentina root extract | 0.1 |
| Tranexamic acid methylamide hydrochloride | 0.1 |
| Paraoxybenzoic acid ester | As |
| | suitable |
| 2-ethylhexyl-para-methoxycinnamate | 1 |
| Red iron oxide | As |
| | suitable |
| Yellow iron oxide | As |
| | suitable |
| Black iron oxide | As |
| | suitable |
| Xanthan gum | 0.1 |
| Sodium acrylate/2-acrylamido-2- | 1 |
| methylpropanesulfonate copolymer | |
| (trade name: Simulgel EG, Seppic) | |
| Bentonite | 1 |
| Flaked inorganic composite powder (talc | 5 |
| coated with titanium oxide, aluminum oxide | |
| and silicic anhydride, trade name: Coverleaf | |
| AR-80, Catalysts and Chemicals Industries) | |
| Sodium carboxymethyl cellulose | 0.1 |
| Purified water | Remainder |
| Fragrance | As |
| | suitable |

### Prescription Example 23

### Foundation

| Prescription | (wt %) |
|---|---|
| Dodecamethylcyclohexasiloxane | 15 |
| Decamethylcyclopentasiloxane | Remainder |
| Pullulan 3-tris(trimethylsiloxy) | 5 |
| silylpropyl carbamate | |
| Dow Corning 9041 Silicone Elastomer Blend | 30 |
| (dimethylpolysiloxane blend of dimethicone | |
| crosspolymer, Dow Corning) | |
| Ethanol | 10 |
| Isostearic acid | 0.5 |
| Myristic acid-treated zinc oxide | 0.5 |
| Dextrin palmitate-coated titanium oxide | 10 |
| Dextrin palmitate-coated talc | 7 |
| Silicone surface-treated titanium | 5 |
| oxide (30 nm) | |
| Crosslinked silicone powder | 1 |
| Spherical silicic anhydride | 2 |
| Magnesium L-ascorbyl phosphate | 0.2 |
| DL-α-tocopherol acetate | 0.1 |
| D-δ-tocopherol | 0.1 |
| Glutathione | 0.1 |
| Valerian extract | 0.1 |
| Tranexamic acid methylamide hydrochloride | 0.1 |
| Shosaikoto extract | 1 |
| Biodyne EMPP | 2 |
| 2-ethylhexyl-para-methoxycinnamate | 5 |
| Dextrin palmitate-coated red iron oxide | As |
| | suitable |
| Dextrin palmitate-coated yellow iron oxide | As |
| | suitable |
| Dextrin palmitate-coated black iron oxide | As |
| | suitable |
| Fragrance | As |
| | suitable |

### Prescription Example 24

### Cosmetic Base

| Prescription | (wt %) |
|---|---|
| Dimethylpolysiloxane | 5 |
| Pullulan 3-tris(trimethylsiloxy) | 0.1 |
| silylpropyl carbamate | |
| Dow Corning 9045 Silicone Elastomer Blend | 30 |
| (decamethylcyclopentasiloxane blend of | |
| dimethicone crosspolymer, Dow Corning) | |
| Decamethylcyclopentasiloxane | Remainder |
| Ethyl alcohol | 8 |
| Prussian blue-coated titanated mica | 0.5 |
| Flaked inorganic composite powder (talc | 5 |
| coated with titanium oxide, aluminum oxide | |
| and silicic anhydride, trade name: Coverleaf | |
| AR-80, Catalysts and Chemicals Industries) | |
| Methylsiloxane network polymer | 5 |
| Crosslinked silicone powder (Torayfil | 5 |
| E-506) | |
| Citric acid | 0.02 |
| Sodium citrate | 0.08 |
| Tocopherol acetate | 0.1 |
| δ-tocopherol | 0.1 |
| Tumeric extract | 0.01 |
| Valerian extract | 0.1 |
| Tranexamic acid methylamide hydrochloride | 3 |
| Biodyne EMPP | 0.1 |
| Purified water | 1 |
| Poly(oxyethylene-oxypropylene)- | 5 |
| methylpolysiloxane copolymer | |

### Prescription Example 25

### Cosmetic Base

| Prescription | (wt %) |
|---|---|
| Dimethylpolysiloxane | 5 |
| Pullulan 3-tris(trimethylsiloxy) | 1 |
| silylpropyl carbamate | |
| (Dimethicone/vinyl dimethicone cross | 15 |
| polymer (trade name: KSG-16, Shin-Etsu | |
| Chemical) | |
| Decamethylcyclopentasiloxane | 25 |
| Polyoxyethylene-methylpolysiloxane | 3 |
| copolymer | |
| Glycerin | 1 |
| 1,3-butylene glycol | 5 |
| Xylitol | 0.5 |
| Isostearic acid | 0.5 |
| Alkyl-modified silicone resin-coated | 2 |
| silicic anhydride | |
| Talc | 0.5 |
| Aluminum stearate | 1 |
| Red iron oxide-coated titanated mica | 0.1 |
| Sodium hexametaphosphate | 0.05 |
| Dipotassium glycyrrhizinate | 0.1 |
| L-serine | 0.1 |
| Lamium Album extract | 0.1 |
| DL-α-tocopherol acetate | 0.2 |
| Thiotaurine | 0.1 |
| Burr rose extract | 0.1 |
| Peony extract | 0.1 |
| Apricot kernel extract | 0.1 |
| Tranexamic acid methylamide hydrochloride | 0.1 |
| Lysolecithin | 0.01 |
| Sodium acetylated hyaluronate | 0.1 |
| Saxifraga sarmentosa extract | 0.1 |
| Biodyne EMPP | 0.1 |
| Paraoxybenzoic acid ester | As |
| | suitable |
| Phenoxyethanol | As |
| | suitable |
| Dextrin palmitate-coated yellow iron oxide | 0.1 |
| Dimethyl distearyl ammonium hectorite | 1 |
| Purified water | Remainder |
| Trimethylsiloxysilicic acid | 1.5 |
| Spherical silicic anhydride | 1 |
| Spherical polyethylene powder | 5 |
| Fragrance | As |
| | suitable |

### Prescription Example 26

### Tablets

| Prescription | (wt %) |
|---|---|
| Microcrystalline cellulose | 35.0 |
| Refined saccharose | 29.0 |
| Carboxymethyl cellulose | 10.0 |
| Cornstarch | 10.0 |
| Talc | 10.0 |
| Polyvinyl pyrrolidone | 5.0 |
| Iris Florentina root extract | 0.1 |
| Tranexamic acid methylamide hydrochloride | 1.0 |

1. A bloating ameliorant, comprising: a tranexamic acid amide derivative represented by the following general formula (I): 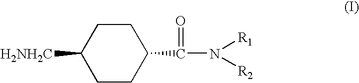
wherein, R₁ and R₂ represent hydrogen atoms, linear or branched alkyl groups having 1 to 18 carbon atoms, cycloalkyl groups having 5 to 8 carbon atoms, benzyl groups or groups represented by the following general formula (II): 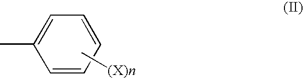
wherein, X represents a lower alkyl group, lower alkoxy group, hydroxy group, amino group or halogen atom and n=0 to 3, and
R₁ and R₂ may be the same or different and/or a salt thereof.

2. A lymphedema ameliorant comprising the tranexamic acid amide derivative and/or salt thereof according to claim 1.

3. A wrinkle ameliorant comprising the tranexamic acid amide derivative and/or salt thereof according to claim 1.

4. An obesity ameliorant comprising the tranexamic acid amide derivative and/or salt thereof according to claim 1.

5. A lymphangiogenesis and/or a lymphatic vessel function promoter comprising the tranexamic acid amide derivative and/or salt thereof according to claim 1.

6. A VEGFC production promoter comprising the tranexamic acid amide derivative and/or salt thereof according to claim 1.
